(19) United States
(12) Patent Application Publication
(10) Pub. No.: US 2009/0286802 A1
(43) Pub. Date: Nov. 19, 2009

(54) High Dose Folic Acid Compositions for Vascular Dysfunction

(76) Inventor: Ahmed Tawakol, Lexington, MA (US)

Correspondence Address: EWARDS ANGELL PALMER & DODGE LLP, P.O. BOX 55874, BOSTON, MA, 02205, US

(21) Appl. No.: 11/918,507

(22) PCT Filed: Apr. 13, 2006
(86) PCT No.: PCT/US2006/013977

§ 371 Date: Jun. 22, 2009

Related U.S. Application Data
(60) Provisional application No. 60/670,961, filed on Apr. 13, 2005.

(51) Int. Cl. A61K 31/519

(52) U.S. Cl. 514/252.16

(57) ABSTRACT

The present invention relates to treatments for hypertension and more particularly, to the use of high dose folic acid in treating dysfunctional conditions associated with vascular function. High dose folic acid can increase vasodilator-stimulated myocardial blood flow and acutely lower arterial pressure independently of homocysteine lowering.

This invention was made with government support under AT16046 awarded by the National Institutes of Health. The Government has certain rights in the invention.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

1. Field of the Invention

The invention relates generally to the use of high dose folic acid in treating the effects of coronary artery disease and dysfunctional conditions associated with deficient vascular function and more particularly to the field of medical treatment for hypertension.

2. Description of Background Art

Coronary heart disease (CHD) is the most common form of heart disease and a major health risk, particularly in the United States where it contributes to over 500,000 deaths each year. Increasing age, heredity and lifestyle can all contribute to CHD, which is estimated to affect at least 12 million Americans in various degrees.

Heart disease is caused by narrowing of the coronary arteries that feed the heart. The heart is a muscle and as such requires a reliable and efficient blood supply to provide oxygen and nutrients. When coronary arteries become narrowed or clogged by fat and cholesterol, CHD results leading to chest pain (angina), arrhythmias, or eventually to a heart attack if the blood supply to a portion of the heart is completely blocked.

Major risks of CHD include uncontrollable factors such as age or sex; however, many risk factors can be eliminated or controlled at least to some extent, such as smoking, obesity, lifestyle, diabetes, cholesterol levels and high blood pressure.

There is a wide range of treatments for heart disease including surgery and drugs. The more drastic procedures range from heart transplants and artificial hearts, pacemakers and implantable cardiac defibrillators to cholesterol-lowering and blood pressure lowering drugs down to drugs designed to alleviate early physical symptoms of CHD.

Drug treatment can in some cases prevent or reduce long-term damage to the heart by lowering blood pressure or cholesterol, and preventing or dissolving blood clots. The major classes of drugs used in treating heart disease are ACE inhibitors, antiplatelet agents, beta blockers, calcium channel blockers, nitrates and salicylates. In particular, ACE inhibitors, beta and calcium channel blockers have become popular drugs for controlling high blood pressure.

Unfortunately, many of the drugs conventionally used to control and treat CHD may have severe side effects; consequently, there are ongoing efforts to improve available drugs and develop new therapies.

Folic acid (also known as vitamin M, folate, folic acid, folacin, pteroylglutamic acid, pteroylmonoglutamic acid) is a well-known nutrient found in natural products, mainly in green leafy vegetables, liver, yeast and fruits and is often used to treat vitamin B deficiency. Oral tablet formulations typically range from 100 mcg up to 1 mg. When used as a dietary supplement, typical amounts range from 400 mg to 0.1 mg as a component in a nutritional supplement (U.S. Pat. No. 6,159,506; U.S. Pat. No. 6,054,128). It is rapidly absorbed from the gastrointestinal tract following oral administration and achieves peak blood levels 30-60 minutes after administration. The metabolically active form of folic acid is tetrahydrofolic acid, but it is also further metabolized in the liver to N⁵-methyltetrahydrofolic acid.

Folic acid has been reported to provide some beneficial effects in the treatment or prevention of stroke or Alzheimer's Disease (U.S. Pat. No. 6,369,058). There are also examples of the use of folic acid in combination with other ingredients for treatments of various conditions. For treating erectile dysfunction, adding a small amount of folic acid to sildenafil citrate formulations was shown to enhance levels of cGMP so that sildenafil doses could be decreased, thereby reducing potential side effects but retaining the effect on sexual response (U.S. Pat. No. 6,338,862). In treating vitiligo, a cutaneous depigmentation disease, high daily doses of 1-50 mg folic acid were administered in order to raise blood levels of folic acid as these patients typically have diminished folic acid levels compared to undiseased patients (U.S. Pat. No. 4,985,443).

Observations from cardiovascular studies involving low dose and dietary folic acid have also been reported in the medical literature. Limited studies on hypertensive male patients provided evidence that 5 mg doses of folic acid may prevent orthostatic dysregultion. (Bechir, M., et al., J. Cardiovasc. Pharmacol. 2005 January; 45(1):44-48). Other studies have noted different effects of antihypertensive drugs on endothelial dysfunction and have named folic acid as among several compounds of possible use in restoring impaired endothelial function. (Puddu, P., et al., Acta Cardiol. 2004 October; 59(5):555-564). Cardiovascular effects of compositions that included low dose folic acid in combination with vitamin C and vitamin E showed a decrease in systolic blood pressure in young, healthy adults but no change in other cardiovascular variables. (Schutte, A. E., et al., Int. J. Vitam. Nutr. Res. 2004 July; 74(4):285-93). In postmenopausal woman, folic acid administered alone in 7.5 mg daily doses resulted in lowered homocysteine levels and an increase in endothelial function. (Paradisi, G., et al., 2004 April; 19(4): 1031-5). In a study of U.S. women, a decreased risk of hypertension was found for younger women who consumed at least 1 mg folate daily. (Forman, J. P., et al., 2005 Jan. 19; 293(3): 320-9.)

3. Deficiencies in the Art

Despite progress in developing therapeutic agents for treating and preventing cardiovascular heart disease, there is a need for interventions and drugs that safely and effectively treat hypertension. Many of the current drugs have significant side effects, while others are contraindicated for older patients or those with diseases such as diabetes or immune dysfunctions. The normal, healthy body tolerates and processes xenobiotics remarkably well, but when under physiological stress of coronary heart disease would ideally respond to treatments that do not require extraordinary metabolism or are enhancements of normal processes.

### SUMMARY OF THE INVENTION

The present invention demonstrates that folic acid can be administered at high doses as an antihypertensive agent. A surprising aspect of using high doses of folic acid is that its vasodilation effect can occur in the absence of an effect on homocysteine levels, which previously were shown to be affected by low doses of folic acid in the range of 1-5 mg. As shown herein, high dose folic acid increases both vasodilator-stimulated myocardial blood flow as well as flow reserve in myocardial segments with impaired dilator function, indicating a clinical role for high dose folic acid in the therapy of ischemic heart disease.

Accordingly, one aspect of the invention is a method for improving vascular dilation in a subject, comprising administering to the subject in need thereof an amount of folic acid or a pharmaceutically acceptable salt, ester, or prodrug thereof comprising about 20 to about 100 mg in a pharmaceutically acceptable vehicle thereby improving vascular dilation in the subject.

In many cases the subject is under care or is known to have some form of coronary heart disease which can be ischemic heart disease. This frequently presents as primary or essential hypertension and most often is associated with older patients, although heart disease has become increasingly prevalent in the middle aged population. However, improvement of vascular dilation may be beneficial for subjects with high blood cholesterol, which is considered a risk factor in the development of coronary heart disease as is high blood pressure, generally defined as equal to or greater than 140/90 mm Hg.

Other risk factors may prompt the use of high doses of folic acid as either a therapeutic measure or a prophylactic intervention. Such risk factors include tobacco smoking, low physical activity and obesity.

The amount of folic acid to be administered should be well above the usual amounts found in standard vitamin formulations or in combinations with other active drugs, for example those drugs used to treat cardiac disease. Preferably, folic acid is to be administered in amounts of about 20 mg up to about 100 mg, either in daily doses at this level or in a single administration. An exemplary effective dose will be a single daily dose of 30 mg or divided doses such as twice daily 15 mg administration.

Administration is most conveniently performed orally but intramuscular, intraveneous or transdermal methods may also be employed. Folic acid is normally readily absorbed in the small intestine, but some patients may not effectively absorb folic acid so that other than oral administration may be indicated in order to achieve desirable blood levels.

It is believed that a sufficiently high dose of folic acid should be administered to sustain a blood level of one or more active metabolites of folic acid. Administration of folic acid can be either as a single daily dose or several doses daily cumulative to at least 20 to 100 mg, but in any event sufficient to maintain a blood level of an active folic acid metabolite, or sufficient storage levels of N⁵-methyltetrahydrofolate to provide a sustained level of active metabolite.

Blood levels of the main active folic acid metabolite (tetrahydrofolate) will preferably be in the range of about 120 to 500 ng/ml subsequent to folic acid administration.

Active folic acid metabolites such as tetrahydrofolate can be directly administered in amounts of about 5 to 50 mg, either in daily doses at this level or in a single administration. An intermediate product of the metabolism of folic acid is folinic acid, which may alternatively be administered to achieve the effects disclosed for high folic acid doses. An exemplary effective folinic acid dose will be a single daily dose of 30 mg or divided doses in twice daily 15 mg administration. Folinic acid calcium salt, commonly known as Leucosar, can be used where it is believed that folic acid conversion to its active metabolite may be blocked; for example, in patients on methotrexate therapy.

Yet another aspect of the invention is a method for increasing nitric oxide bioavailability in a subject comprising administering to the subject about 20 to about 100 mg of folic acid. This will produce an increase in adenosine-induced blood flow resulting in increased nitric oxide bioavailability. A preferred amount of folic acid is about 30 mg, administered as previously described and in any event in an amount sufficient to increase adenosine-induced blood flow with increased nitric oxide bioavailability.

The new treatment method may also be employed for reducing systemic blood pressure, particularly in patients with high blood pressure. Thus a subject needing to have a reduction in blood pressure will be administered about 30 mg of folic acid or a dose of folic acid from about 20 to about 100 mg which will reduce diastolic blood pressure by at least about 5 mm Hg. Where desirable, the amount of folic acid administered may be such as not to cause a significant change in homocysteine blood levels.

An aspect of the invention that did not become apparent until the effect of high dose folic acid on vasodilator function was observed, is the use of high dose folic acid in treating erectile dysfunction. Accordingly, high dose folic acid can be an alternative to the erectile dysfunction preparations currently on the market as prescription drugs and should have a beneficial effect on females as well as males. Additionally, it can be used safely by patients with coronary artery disease without fear that systemic dilation will preclude use for erectile treatments.

Accordingly, another aspect of the invention is a method for treating sexual dysfunction. The method comprises administering to a subject a composition consisting of an effective amount of folic acid or a folic acid active metabolite to thereby treat the sexual dysfunction. The treatment is applicable to sexual dysfunction in males and females. Of particular prevalence is erectile dysfunction and impotence in males, while females can benefit from treatment of orgasmic dysfunction and sexual arousal. In both sexes, the disclosed folic acid treatment is expected to address conditions where arterial circulation is insufficient or impaired.

The effective amount of folic acid for treating sexual dysfunction is expected to be between about 15 mg to about 100 mg. Sexual dysfunction may be a treatable problem in a wide range of age groups in healthy as well as those with diseases or conditions that affect the vascular system. Accordingly, the amount of high dose folic acid will be carefully assessed on a case by case basis, taking into account the age and health of the subject and preferably monitoring blood levels of active metabolite with respect to functional improvement in order to determine optimal amounts of high dose folic acid.

Other aspects of the invention comprise compositions that include high dose folic acid. A preferable composition is one containing about 30 mg folic acid in a pharmaceutically acceptable vehicle, but other high dose folic acid formulations are contemplated, such as about 40, 50, 60, 70, 80, 100 or several hundred mg preparations.

Folic acid compositions may additionally include a selected drug suitable for improving vascular dilation, particularly for conditions related to coronary heart disease. Such selected drugs include ACE inhibitors, beta blockers, calcium channel blockers, nitrates and salicylates. In certain instances other drugs may be used in combination with high dose folic acid, including diuretics, cholesterol and triglyceride lowering drugs, anti-thrombotic agents and antiplatelet drugs.

High dose folic acid compositions may be formulated as orally acceptable tablets, preferably as single 30 mg tablets. The tablets will be formulated by well-known procedures to render the tablets suitable for oral administration, conventionally including an orally acceptable dispersant and/or ingredients that enhance absorption.

Oral formulations of high dose folic acid can also be in the form of a sustained release tablet that provides a blood level of an active metabolite of folic acid, such as 5-methylene tetrahydrofolate that is effective in reducing diastolic blood pressure by at least 5 mm Hg. Such formulations will normally be used in patients suffering from coronary heart disease.

As discussed, folic acid or its intermediary or active metabolites, can be administered by intraveneous, transdermal, or other means that provide the agent to the bloodstream by other than oral absorption. Such alternative methods of administration may be desirable in certain groups of young patients or old patients or when conditions exist that inhibit or interfere with normal absorption through the intestine.

Packaged formulations containing high doses of folic acid and instructions for use are also within the scope of the invention. Thus such formulations will be provided for use in reducing systemic blood pressure, treating sexual dysfunction or for use with any of the methods disclosed and described herein.

The high dose folic acid compositions of the invention can also be useful prophylactically in preventing or delaying the onset of the diseases associated with vascular insufficiency. Thus high doses of folic acid can prevent abnormal elevation of blood pressure and assist in delaying the development of coronary heart disease.

Other aspects of the invention are described in or are obvious from the following disclosure, and are within the ambit of the invention.

### BRIEF DESCRIPTION OF THE FIGURES

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings in which:

FIG. 1. Effect of High-Dose Folate on Mean Arterial Pressure. Resting mean arterial blood pressure (MAP) was measured. Individual responses are shown and group mean data (±SD) are depicted by the thick line. Folate significantly reduced MAP (100±3 vs. 96±2 mmHg, placebo vs. folate, P<0.03).

FIG. 2. Effect of High-Dose Folate on Peak (Adenosine-stimulated) Myocardial Blood Flow Peak adenosine-stimulated myocardial blood flow (MBF) was measured in ABNORMAL zones. Individual responses are shown and group mean data are depicted by the thick line. Folate significantly increased peak MBF (1.45±0.59 vs. 2.16±1.01 ml/min/gm, mean±SD, placebo vs. folate, P<0.02).

FIG. 3. Effect of High-Dose Folate on the Coronary Dilator Reserve Dilator reserve was measured in NORMAL (WNL) and ABNORMAL (ABNL) regions. Mean data and standard error bars are depicted. Folate increased dilator reserve by ˜83% in ABNORMAL segments (0.72±0.60 vs. 1.31±1.08 ml/min/gm, mean±SD, placebo vs. folate, P<0.05), while dilator reserve in NORMAL segments remained unchanged (2.00±0.61 vs. 2.12±0.69 ml/min/gm, placebo vs. folate, P=NS).

FIG. 4. Effect of High-Dose Folate on Peak Flow Ratio. The ratio for peak MBF in ABNORMAL relative to NORMAL segments, is shown for each individual. Group mean data are depicted by the thick line. Folate increased the ratio of flow in ABNORMAL segments relative to NORMAL segments (0.54±0.17 vs. 0.75±0.24, mean±SD, placebo vs. folate, P<0.01).

### DETAILED DESCRIPTION OF THE INVENTION

### 1. Definitions

“Vascular dilation” (also known as vasodilation) is relaxation of blood vessels, a condition that typically occurs in order to allow for increased blood flow to the vascular bed in need of additional blood. Abnormal vasodilation commonly develops in humans, and results in hypertension, and is also a critical component of coronary artery disease.

“Folic acid” (folate) is a B-vitamin that is required for transmethylation reactions, nucleic acid synthesis, homocysteine metabolism, and the enzymatic regeneration of tetrahydrobiopterin, an essential co-factor of nitric oxide synthase (Verhaar, M. C. 5-methyltetrahydrofolate, the active form of folic acid, restores endothelial function in familial hypercholesterolemia. Circulation 1998; 97:237-4). Folic acid is identified under the chemical names N[4-[[(2-amino-1,4-dihydro-4-oxo-6-pteridinyl)methyl]amino]benzoyl]-L-glutamic acid; or N-(p-[(2-amino-4-hydroxy-6-pteridinyl)methyl]amino]benzoyl]-glutamic acid; pteroylglutamic acid; or N-(p-[2-amino-4-hydroxypryimido[[4,5b]pyrazin-6-yl)methylamino]benzoyl-glutamic acid. Folic acid is a coenzyme precursor that converts to tetrahydrofolate in the body.

“Metabolite” as used herein is in reference to one or more metabolites of folic acid. Metabolites generated in successive biochemical reactions may be referred to as intermediate metabolites. It will be recognized that some metabolites are active while others do not exhibit activity associated with folic acid. The major active metabolite of folic acid is tetrahydrofolate. Another active metabolite is folinic acid is N-[4-[[2-amino-5-formyl-1,4,5,6,7,8-hexahydro-4-oxo-6-pteridinyl)methyl]amino]benzoyl]-L-glutamic acid, also known as citrovorum factor and leucovorin. Leucovorin calcium is a calcium salt of folinic acid.

Several additional active metabolites are related to folate metabolism. Important amongst them are the biopterins, whose regeneration requires folate. Biopterin is 2-amino-4-hydroxy-6-(1,2-dihydroxypropyl)pteridine. Another biopterin is tetrahydrobiopterin, which is a necessary co-factor for the generation of nitric oxide (via nitric oxide synthase). As used herein, the biopterins will also be referred to as “folate metabolites.”

“Blood pressure” refers to peripheral blood pressure conventionally measured as mmHg and reflects the pressure of the blood against the inner walls of the arteries. The top number, or systolic blood pressure (SBP) reflects the pressure in the arteries when the heart is pumping. The bottom number or the diastolic blood pressure (DBP) represents the arterial pressure when the heart is resting. Blood pressure is usually reported as systolic/diastolic using terminology such as 130 over 83.

“Homocysteine” is an amino acid normally present in human blood. Elevated levels are considered a sign of increased risk for heart attack.

A “Prodrug” is an inactive precursor of a drug, converted into its active form in the body by normal metabolic processes.

“Sexual dysfunction,” as used herein, can include any sexual dysfunction in an animal, preferably human and can be either male or female. Sexual dysfunctional disorders may include arousal or orgasmic disorders. In females this may be associated with pre-menopausal or menopausal disorders, vaginismus or sexual pain. Particularly in males there may be problems with erectile dysfunction or impotence.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Use of the term “a” in claims or otherwise in the specification is not intended to be limiting to the singular form except where specifically indicated such as in the form of enumeration; e.g., “one” or “single” and like terms.

### 2. Pharmaceutical Compositions

Pharmaceutical compositions and dosage forms of the invention comprise high dose folic acid, or metabolites thereof, including the biopterin family, alone or in combination with other active ingredients in relative amounts and formulated so that a given pharmaceutical composition or dosage form is effective in treating hypertension. Preferred pharmaceutical compositions and dosage forms comprise folic acid or its active metabolite or pharmaceutically acceptable salts, solvates, prodrugs or clathrates thereof, optionally in combination with one or more additional active agents.

Compositions containing the disclosed high dose forms of folic acid may be administered in several ways, including orally, parenterally, intraperitoneally, intradermally or intramuscularly. Pharmaceutical forms suitable for injection include sterile aqueous solutions or dispersions for extemporaneous preparation of the solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained by the use of a coating such as lecithin, by the maintenance of the required particle size in case of a dispersion and by the use of surfactants. The prevention of the action of microorganisms can be effected by various antibacterial and antifungal agents such as parabens, chlorobutanol, phenol, sorbic acid, thimerosal and the like. In many cases, isotonic agents may be included, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral dosage forms are also contemplated and particularly preferred. Pharmaceutical compositions of the invention suitable for oral administration can be presented as discrete dosage forms, including but not limited to, tablets (e.g. chewable tablets), caplets, capsules and liquids such as flavored syrups. Dosage forms containing predetermined amounts of active ingredients may be prepared by well known methods of pharmacy, see Remington's Pharmaceutical Sciences (1990) 18^{th} ed., Mack Publishing Co., Easton, Pa.

Typical oral dosage forms of the invention are prepared by combining the active ingredient(s) in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivates (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. One specific binder is a mixture of microcrystalline cellulose and sodium carboxymethyl cellulose sold as AVICEL RC-581. Suitable anhydrous or low moisture excipients or additives include AVICEL-PH-103J and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferable from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crosprovidone, polacrilin potassium, sodium starch glycolate, potato or tapioca starch, other starches, pre-gelatinized starch, other starches, clays, other algins, other cellulosses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The pH of a pharmaceutical composition or dosage form, or of the tissue where the composition or dosage form is applied, may be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients to improve delivery. Stearates for example can serve as a lipid vehicle for the formulation, as an emulsifying agent or surfactant, and as a delivery-enhancing or penetration-enhancing agent. Salts, hydrates or solvates of the active ingredients can be used to further adjust the properties of the resulting compositions.

Upon formulation, solutions will be administered in a manner compatible with the dosage formulation and in such amount as is therapeutically effective. The formulations are easily administered in a variety of dosage forms preferably as injectable solutions.

It is not believed that there is a particular high dose of folic acid that may be beneficial for coronary artery disease treatments. While beneficial results have been shown with single daily 30 mg doses, any dose about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84,85, 86, 87, 88, 89, 90 up 100 mg or even to several hundred mg may be beneficial in some cases. Care should be taken to consider the age, health and condition of the subject as long as the doses remain an order of magnitude below the LD50. Ideally, blood levels of the active metabolite of folic acid should be taken for each patient as dose is adjusted in accordance with the desired effect; e.g., lowering of blood pressure. It may be preferable to monitor the active metabolite levels as well as folic acid levels in the blood in order to optimize dosing.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intradermal and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

The present invention is additionally described by way of the following illustrative, non-limiting Examples that provide a better understanding of the present invention and of its many advantages.

### EXAMPLES

Low dose administration and dietary supplementation of folic acid has gained attention as a potential treatment of hyperhomocysteinemia in patients with atherosclerotic disease (Boushey, C. J., et. al., JAMA 1995, 274(13):1049-57; Chambers, J. C., et al., Circulation 2000; 102(20) 2479-83). It is also recognized that low dose folic acid has a therapeutic effect on vascular endothelial function (Stroes, E. S., et al., Circ Res 2000; 86(11):1129-1134, Doshi, S. N., et al., Circulation 2002; 105(1):22-26; Doshi, S. N., et al., Art Thromb Vasc Biol 2001; 21(7):1196-1202; Usui, M., et al., Clinical Science 1999; 96:235-239).

In an effort to determine whether or not folic acid can acutely improve coronary dilation in patients with ischemic heart disease, a double-blinded, placebo-controlled cross-over study was conducted. As shown herein, high dose folic acid improved coronary dilator function in patients with coronary artery disease. To assess the direct vascular effect of folic acid independently of its homocysteine lowering effect, patients with normal homocysteine concentrations were studies and myocardial blood flow acutely after ingestion of folate measured before homocysteine-lowering occurred.

Also shown are data of the effect of folate (30 mg) on myocardial blood flow. The magnitude of the effect of folate (30 mg) was similar to that of sildenafil (100 mg).

The examples described herein indicate that folic acid acts to reduce blood pressure in human subjects due to enhanced endothelial function and resulting systemic vasodilatation. Given its minimal side effects and other therapeutic benefits for patients with atherosclerotic disease, folic acid is expected to be a low-risk and inexpensive option for treatment of hypertension and sexual dysfunction.

### Example 1

### High Dose Folic Acid Patient Studies

The following abbreviations are used: Ado 70=adenosine 70 μg/kg/min; Ado 140=adenosine 140 μg/kg/min; MBF=myocardial blood flow; PET=Positron Emission Tomography.

In this example, a clinical study was conducted showing that high-dose folic acid increases both vasodilator stimulated MBF as well as flow reserve in myocardial segments with impaired dilator function and acutely lowers arterial pressure independently of homocysteine lowering. These results document the effects of high dose in the peripheral circulation, the coronary circulation, and highlight a potentially useful clinical role for folic acid in the therapy of ischemic heart disease.

14 patients with ischemic heart disease, age 62±12yr. (mean±SD), were enrolled in a double-blind, placebo controlled crossover trial. Basal and adenosine-stimulated myocardial blood flow (MBF) were determined by PET and myocardial flow reserve was calculated. Each patient was studied after ingestion of placebo and after ingestion of 30 mg folic acid. Myocardial zones were prospectively defined physiologically as “NORMAL” vs “ABNORMAL” based on MBF response to Ado 140 (NORMAL=MBF>1.65 ml/min/g). ABNORMAL and NORMAL zones were analyzed separately in a patient based analysis.

Patient Population: Adult male and female patients with coronary artery disease and normal serum homocysteine concentration (HCY<12 μM) were recruited from the greater Boston area. Coronary artery disease was defined as the presence of a >50% stenosis in at least one coronary artery identified by coronary arteriography within the last 5 years. Exclusion criteria included: inability to maintain a stable medical regimen during the study period, concurrent use of folic acid, or myocardial infarction or coronary intervention within the proceeding 3 months. The study protocol was approved by the local Human Research Committee and informed consent was obtained from each subject.

Study Drug Administration. Subjects were enrolled in a double-blinded, placebo-controlled, crossover study during which folate syrup (30 mg) or similar tasting placebo syrup was administered orally in two divided doses, 10-12 hours and 1 hour prior to MBF measurements. The 1-hr time point and the dose of 30 mg were chosen to achieve a similar peak plasma 5-methyltetrahydrofolate level (Perry, J., et al., Br J Haematol 1970; 18:329-39) (the biologically active form of folic acid) that has previously been shown to improve peripheral nitric oxide-mediated vasodilation (Verhaar, M. C., Circulation 1998; 97:237-41). Folate and placebo were administered at least one week apart, in random order, and in a double-blinded manner.

Myocardial Blood Flow Measurements. Vasoactive medications were withheld for 3-5 half-lives, and subjects were asked to avoid caffeinated beverages for 24 h before blood flow measurements. MBF was assessed with PET (GE Medical Systems Scanditronix PC4096), approximately 1-hr after ingestion of the second dose of the study drug. PET measurements of MBF (¹³N-ammonia method) were performed at rest and during the infusion of two doses of adenosine (70 and 140 mcg/kg/min), using a previously described method (Huggins, G. S., et al., Circulation 1998; 98:1291-6).

PET Image Analysis: Three short axis rings corresponding to the proximal, middle and distal thirds of the left ventricle were constructed for each K1 scan and MBF was measured within 24 standard areas of interest as described previously (Huggins, G. S., et al., Circulation 1998; 98:1291-6). Data analysis was performed without knowledge of treatment order. To determine the variability of the MBF measurement, two readers independently assessed adenosine-stimulated MBF in 120 segments in 5 subjects and the difference between and within readers was calculated. The mean (±SD) intra-, and inter-observer difference was 0.03±0.10 and 0.03±0.17 ml/min/gm, respectively.

Prospective Definition of Myocardial Zones. MBF in regions supplied by stenotic conduit vessels, which were expected to have abnormal dilator capacity, were prospectively examined separately from regions with normal dilator capacity. Data demonstrated that maximal MBF>1.65 ml/min/g with high dose adenosine has very high negative predictive accuracy (91%) for exclusion of moderate to severe coronary artery stenosis and that 97% moderate to severe stenoses had maximal MBF<1.65 ml/min/g (5). Accordingly, myocardial zones were defined physiologically as “NORMAL” vs. “ABNORMAL” based on MBF response to Ado 140 during the placebo condition. Myocardial zones with MBF with Ado 140 of <1.65 ml/min/g during placebo condition were defined as ABNORMAL. The corresponding myocardial regions were identified during the folate condition, and were labeled ABNORMAL regardless of their MBF during the folate condition. Values for ABNORMAL MBF were combined and averaged to obtain a single value of ABNORMAL blood flow for each patient at each condition, (placebo and folic acid) for each scan acquisition (rest, adenosine 70, or adenosine 140). A patient-based analysis of MBF was performed.

Dilator reserve for both NORMAL and ABNORMAL zones was defined as the difference between peak MBF and rest MBF. Peak MBF was defined as the higher of the two average MBF values obtained during adenosine (i.e, the greater of the average MBF obtained during adenosine 70 vs. adenosine 140 dose). This was done, since in patients with IHD, coronary “steal” may occur (Holmvang, G., et al., Circulation 1999; 99:2510-6). and so cause underestimation of dilator reserve in ABNORMAL regions.

Coronary Angiography. Coronary angiograms were reviewed in blinded fashion by an expert for the presence and grade of collaterals as previously described (Holmvang, G. et al., Circulation 1999; 99:2510-6.).

Statistical Analysis. Data are expressed as mean±SD. A repeated measures ANOVA was performed, (Statview v 4.0, Abacus Concepts), with terms for the order of folate vs. placebo administration, patient, and observations (blood pressure, MBF and G) for the three repeated measurements (adenosine 0, 70, and 140). To control for multiple testing, prospectively defined pair-wise t-tests comparing folate to placebo at each of the three adenosine doses were performed only if there was a significant main effect for folate or a significant interaction of folate with adenosine in the ANOVA analysis. Values of P<0.05 were considered significant.

Vasodilator Effect of Folic Acid. Prior studies have demonstrated that folate enhances nitric oxide bioavailability (Chambers, J. C., Circulation (Online) 2000; 102:2479-8; Doshi, S. N., et al., Arterioscler Thromb Vasc Biol 2001; 21:1196-202). Nitric oxide appears to play a significant role in adenosine-induced vasodilation in the coronary microcirculation (Tawakol, A., et al., J Am Coll Cardiol 2002; 40:1051-58). The results disclosed herein indicate that the increase in adenosine-induced blood flow occurring after folate ingestion is also a result of increased nitric oxide bioavailability.

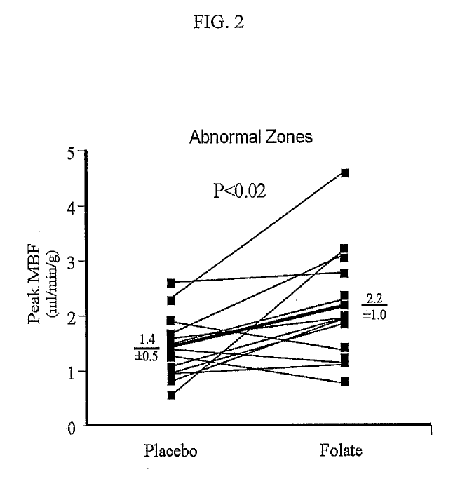

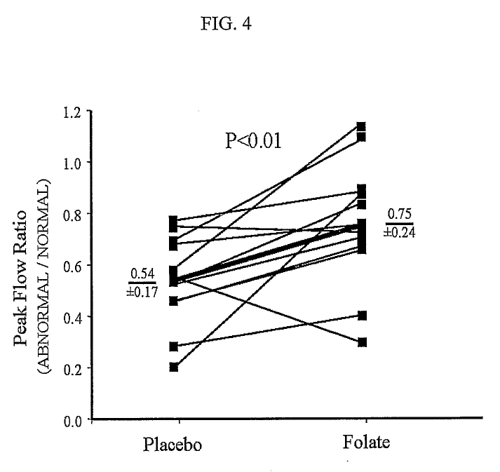

Vascular Locus of Folate's Effects. Both MBF (FIG. 2) and flow reserve (FIG. 4) increased in regions with abnormal flow reserve after folate, a finding that is all the more noteworthy, since diastolic blood pressure (coronary perfusion pressure) was significantly lower with folate (Table 2). While the exact vascular locus of folate' s effects cannot be determined from the results of this study, myocardial dilator capacity is known to be impaired in collateral dependent areas (Sellke, F. W., et al., Circulation 1990; 81:1938-47) in regions supplied by stenotic conduit vessels (Gewirtz, H., Cardiology 1997; 88:62-70), and in resistance vessels distal to a stenosis (Merkus, D., et al., Am J Physiol Heart Circ Physiol 2001; 280:H1674-1682; Fedele, F. A., et al., Circulation 1988; 78:729-35). Thus, the potential for folate to improve vasomotion exists for any of these vessels.

Folate Reduces Systemic Blood Pressure. Long-term low dosage folate (4 weeks to 2 years) has been associated with homocysteine lowering (Mangoni, A. A., et al., J Intern Med 2002; 252:497-503; van Dijk, et al., Arterioscler Thromb Vasc Biol 2001; 21:2072-2079). In the current study, a significant reduction in blood pressure after the ingestion of high-dose folate in 11/14 patients occurred without changes in homocysteine concentration. This suggested that the reduction in systemic blood pressure observed after folate administration may result from enhanced nitric oxide bioavailability.

Clinical Implications. A 5 mm Hg reduction in diastolic blood pressure was observed with folate. Prior meta-analyses have demonstrated that a 5-6 mm Hg reduction in DBP is clinically significant, and is associated with significant reductions in stroke (Collins, R., et al., Lancet 1990; 335:827-38; Rodgers, A., et al., BMJ 1996; 313:147) and heart disease (Collins, R., et al., Lancet 1990; 335:827-38). Long-term use of folate may show sustained effects in lowering blood pressure, demonstrating that it will be a valuable addition to anti-hypertensive therapy.

A significant increase in flow reserve was also observed. Since abnormalities in MBF and flow reserve are associated with manifestation of ischemia in patients with coronary artery disease (Schutte, A. E., et al., Int. J. Vitam. Nutr. Res. 2004 July; 74(4):285-93) it follows logically that high-dose folate is expected to reduce the occurrence of ischemia in patients with coronary disease. Currently there are only limited data to demonstrate that pharmacologic interventions that acutely improve MBF and flow reserve have a significant long-term effect on angina. Accordingly, the potential for folate mediated cardiovascular benefits will address one of the major health problems facing many developed nations.

The current study demonstrates that folic acid has acute vasodilating actions that occur in the absence of homocysteine-lowering. Accordingly, the findings of the current study suggest that doses of folate that are significantly higher than employed in prior trials may impart additional cardiovascular benefits, and, importantly, that the vascular benefits of folic acid may extend beyond its homocysteine-lowering effect.

This study demonstrates that oral folate acutely enhances coronary dilation and modestly lowers arterial pressure in the humans with coronary artery disease. These findings extend observations of effects of folate made in the peripheral circulation to the coronary circulation. Additionally, this study demonstrates an effect of folic acid on vascular function that is independent of its effect on homocysteine-lowering, and raises the possibility that administering doses of folic acid that are higher than previously employed would be expected to confer additional clinical benefits.

A total of 14 patients were studied. Subject characteristics are displayed in Table 1.

TABLE 1
Subject Characteristics (mean ± SD)

| Characteristic | Value |
|---|---|
| Gender (M/F) | 13/1 |
| Age (yr) | 62 ± 12 |
| Diabetes (% subjects) | 20 |
| Smoking (% subjects) | 57 |
| Current | 7 |
| Former | 50 |
| SBP (mmHg) | 125 ± 12 |
| DBP (mmHg) | 74 ± 7 |
| Total Chol.(mg/dL) | 155 ± 30 |
| LDL (mg/dL) | 88 ± 27 |
| HDL (mg/dL) | 41 ± 8 |
| Triglycerides (mg/dL) | 130 ± 79 |
| Statin Use (% subjects) | 100 |
| Beta Blocker use (% subjects) | 80 |
| Calcium Antagonist use (%) | 7 |
| Homocysteine (μmol/L) | 8.7 ± 1.5 |
| Coronary Disease (% subjects) | |
| 1 Vessel | 29 |
| 2 Vessel | 21 |
| 3 Vessel | 50 |
| Prior CABG (% subjects) | 43 |
| Prior MI (% subjects) | 36 |

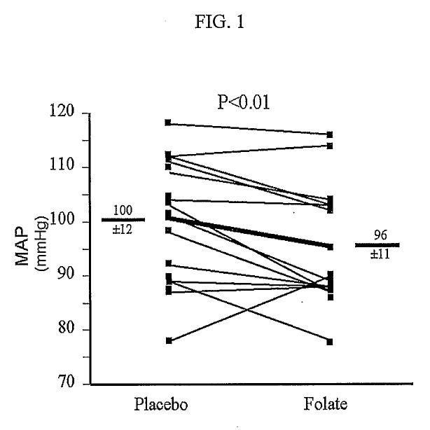

Hemodynamic parameters are displayed in Table 2 and FIG. 1.

TABLE 2
Effect of Folate on Hemodynamic Variables (mean ± SD)

| | | |
|---|---|---|
| Placebo | Folate | P-value |

HR (bpm)

| | | | |
|---|---|---|---|
| Rest | 61 ± 9 | 61 ± 8 | NS |
| Ado 70 | 68 ± 13 | 70 ± 14 | NS |
| Ado 140 | 82 ± 13 | 82 ± 15 | NS |

RPP (mmHg × bpm)

| | | | |
|---|---|---|---|
| Rest | 7617 ± 1717 | 7483 ± 1798 | NS |
| Ado 70 | 9074 ± 2639 | 8604 ± 2484 | NS |
| Ado 140 | 10444 ± 2156 | 9998 ± 3066 | NS |

*SAP (mmHg)

| | | | |
|---|---|---|---|
| Rest | 125 ± 18 | 121 ± 15 | NS |
| Ado 70 | 131 ± 21 | 123 ± 18 | NS |
| Ado 140 | 127 ± 14 | 120 ± 21 | NS |

*DBP (mmHg)

| | | | |
|---|---|---|---|
| Rest | 75 ± 9 | 70 ± 9 | 0.01 |
| Ado 70 | 74 ± 12 | 68 ± 11 | 0.04 |
| Ado 140 | 75 ± 12 | 69 ± 10 | 0.01 |

*MAP (mmHg)

| | | | |
|---|---|---|---|
| Rest | 100 ± 12 | 96 ± 11 | 0.03 |
| Ado 70 | 103 ± 15 | 95 ± 13 | 0.01 |
| Ado 140 | 101 ± 14 | 95 ± 15 | 0.03 |

*P < 0.05 ANOVA for adenosine dose response curve (placebo vs. folate conditions)
Abbreviations used:
Ado 70 = adenosine 70 μg/kg/min
Ado 140 = adenosine 140 μg/kg/min
HR = heart rate
RPP = rate pressure product (SAP × HR)
SAP, DAP, and MAP = systolic, diastolic, and mean blood pressure, respectively

There was no significant change in HR or RPP (folate vs. placebo). However, folate caused a significant reduction in systolic, diastolic and mean arterial blood pressures (P<0.01 ANOVA, Table 2). An order effect for placebo vs. folate was never significant for any of the analyses of variance for the hemodynamic (or any other) measurements.

Folate did not affect myocardial blood flow in NORMAL regions (Table 3, FIGS. 2-4).

TABLE 3
Effect of Folate on Myocardial Blood Flow (mean ± SD)

| | | |
|---|---|---|
| Placebo | Folate | P-value |

Myocardial Blood Flow (ml/min/gm)
*ABNORMAL

| | | | |
|---|---|---|---|
| Rest | 0.68 ± 0.24 | 0.74 ± 0.27 | NS |
| Ado 70 | 1.30 ± 0.71 | 1.60 ± 1.15 | NS |
| Ado 140 | 1.18 ± 0.32 | 1.76 ± 0.75 | 0.03 |
| Peak | 1.45 ± 0.59 | 2.16 ± 1.01 | 0.02 |

NORMAL

| | | | |
|---|---|---|---|
| Rest | 0.84 ± 0.29 | 0.81 ± 0.19 | NS |
| Ado 70 | 2.10 ± 0.97 | 2.01 ± 0.88 | NS |
| Ado 140 | 2.78 ± 0.67 | 2.72 ± 0.74 | NS |
| Peak | 2.84 ± 0.61 | 2.86 ± 0.67 | NS |

*Ratio ABN:WNL

| | | | |
|---|---|---|---|
| Rest | 0.80 ± 0.15 | 0.95 ± 0.17 | 0.03 |
| Ado 70 | 0.67 ± 0.16 | 0.84 ± 0.37 | NS |
| Ado 140 | 0.47 ± 0.15 | 0.67 ± 0.21 | 0.02 |
| Peak | 0.54 ± 0.17 | 0.75 ± 0.24 | 0.01 |

Dilator Reserve (ml/min/gm)

| | | | |
|---|---|---|---|
| ABNORMAL | 0.77 ± 0.59 | 1.41 ± 1.08 | 0.04 |
| NORMAL | 2.00 ± 0.61 | 2.12 ± 0.69 | NS |
| Ratio ABN:WNL | 0.43 ± 0.21 | 0.63 ± 0.36 | 0.01 |

*P < 0.05 ANOVA for adenosine dose response curve (placebo vs. folate conditions)
Abbreviations:
Ratio = ABNORMAL/NORMAL zone MBF
NORMAL = Myocardial segments with normal dilator capacity during placebo condition
ABNORMAL = Myocardial segments with abnormal dilator capacity during placebo condition
Ado 70 = adenosine 70 μg/kg/min
Ado 140 = adenosine 140 μg/kg/min
MBF = myocardial blood flow

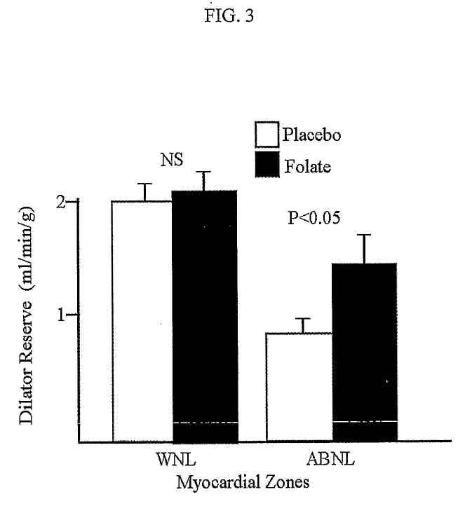

However, in ABNORMAL zones, despite the fall in MAP, folic acid significantly increased the MBF response to adenosine (P<0.001 ANOVA, Table 3). This was accompanied by a 49% increase in peak MBF in ABNORMAL zones (1.45±0.59 vs. 2.16±1.01 ml/min/gm, placebo vs. folate p<0.02, Table 3 and FIG. 2). Furthermore, folate increased dilator reserve by 83% in ABNORMAL segments (0.77±0.59 vs. 1.41±1.08 ml/min/gm, placebo vs. folate, P=0.04, Table 3 and FIG. 3), while dilator reserve in NORMAL segments remained unchanged (2.00±0.61 vs. 2.12±0.69 ml/min/gm, placebo vs. folate, P=NS).

The ratio of MBF in ABNORMAL vs. NORMAL zones was determined for each patient during each condition. There was a significant improvement in ABNORMAL:NORMAL MBF ratio at rest and during adenosine after folate (Table 3). Notably, peak MBF increased in the ABNORMAL zones relative to the NORMAL zones in 85% of patients (FIG. 4). Similarly, flow reserve increased in the ABNORMAL zones relative to the NORMAL zones in 83% of patients.

Coronary Anatomy: Ninety-four percent of myocardial zones that were classified as ABNORMAL by PET were found to be sub-served by stenotic coronary arteries (>70% stenoses). Collaterals of varying grade (I-III) were observed supplying the abnormal zones in 9/13 patients. Of the 10 patients in whom ABNORMAL zone MBF improved after folate, 8/10 had collaterals to the ABNORMAL zone. In contrast, of 3 patients in whom ABNORMAL zone MBF failed to improve after only ⅓ exhibited collaterals to the ABNORMAL zone. Sample size was insufficient to determine if peak MBF response correlated with collateral grade.

Biochemical Data: Total plasma folate levels increased significantly after folic acid ingestion (20±6 vs. 473±106 ng/ml, placebo vs. folate, P<0.01). Conversely, plasma homocysteine concentrations did not change after folic acid (7.1±1.4 vs. 7.8±1.1 μmol/L, placebo vs. folate, P=NS).

Results: Folate was associated with a reduction in mean arterial pressure in (100±12 vs. 96±11 mmHg, placebo vs. folate, P<0.03). Despite the fall in MAP, folic acid significantly increased the MBF dose response to adenosine (P<0.001 ANOVA) in ABNORMAL zones, whereas MBF in NORMAL zones did not change. In ABNORMAL segments, folic acid increased peak MBF by 49% (1.45±0.59 vs. 2.16±1.01 ml/min/gm, p<0.02). Furthermore, folate increased dilator reserve by 83% in ABNORMAL segments (0.77±0.59 vs. 1.41±1.08 ml/min/gm, placebo vs. folate, P<0.05), while dilator reserve in NORMAL segments remained unchanged (2.00±0.61 vs. 2.12±0.69 ml/min/gm, placebo vs. folate, P=NS).

The data demonstrate that high dose oral folate acutely lowers blood pressure

### Example 2

### Comparison of High-Dose Folic Acid and Sildenafil

The effects of sildenafil on myocardial blood flow were assessed. The chemical name for sildenafil is 5-[2-ethoxy-5(-4-methyl piperazine-1-ylsulfonyl)phenyl]-1-methyl-3-propyl-1,6-dihydro-7H-pyrazolo[4,3-d]pyrimidin-7-one. A study design similar to that used to test the effects of folate as described herein was used. In brief, myocardial blood flow (MBF) was measured in 14 patients with stable chronic ischemic heart disease using positron emission tomography with ¹³N-ammonia. Patients were studied in double blind, placebo control, cross-over design with 100 mg sildenafil (or placebo) given ˜2-3 hrs prior to measurements of hemodynamics and myocardial blood flow at rest and during administration of adenosine at (140 μg/kg/min). Sildenafil improved myocardial blood flow in abnormal zones (Table 4). Also shown are data of the effect of folate (30 mg) on myocardial blood flow. The magnitude of the effect of folate (30 mg) was similar to that of sildenafil (100 mg).

TABLE 4
Effect of Folate vs Sildenafil on Myocardial Blood Flow (mean ± SD)

| | | |
|---|---|---|
| Placebo | Intervention | P-value |

Myocardial Blood Flow (ml/min/gm)
Folate

| | | | |
|---|---|---|---|
| Rest | 0.68 ± 0.24 | 0.74 ± 0.27 | NS |
| Ado 140 | 1.18 ± 0.32 | 1.76 ± 0.75 | <0.05 |

Sildenafil

| | | | |
|---|---|---|---|
| Rest | 0.76 ± 0.48 | 0.64 ± 0.20 | NS |
| Ado 140 | 1.20 ± 0.33 | 1.58 ± 0.55 | <0.05 |

Ado 140 = adenosine 140 μg/kg/min
MBF = myocardial blood flow

1. A method for improving vascular dilation in a subject in need thereof, comprising administering to the subject (1) an amount of folic acid or a pharmaceutically acceptable salt, ester or prodrug thereof comprising about 20 to about 100 mg in a pharmaceutically acceptable vehicle or (2) an amount of an active or intermediate metabolite of folic acid or a pharmaceutically acceptable salt, ester or prodrug thereof comprising about 5 to about 50 mg in a pharmaceutically acceptable vehicle, thereby improving vascular dilation in the subject.

2. The method of claim 1 wherein the subject exhibits coronary heart disease.

3. The method of claim 2 wherein the coronary heart disease is ischemic heart disease.

4. The method of claim 1 wherein the administering is performed at least twice daily in a dose of about 15 mg folic acid.

5. The method of claim 1 wherein the administering is performed at least once daily in a dose of about 30 mg.

6. The method of claim I wherein the folic acid is administered at a frequency sufficient to achieve a sustained blood level of tetrahydrofolate.

7. The method of claim 1 wherein the administering is oral.

8. The method of claim 1 wherein the administering is intramuscular, intravenous or transdermal.

9. The method of claim 6 wherein the blood level of tetrahydrofolate is about 250 to about 500 ng/ml.

10. A method for increasing nitric oxide bioavailability in a subject comprising administering to the subject about 30 mg of folic acid to increase adenosine-induced blood flow and thereby increase nitric oxide bioavailability in the subject.

11. The method of claim 1, comprising administering to the subject an amount of about 30 mg of folic acid, thereby reducing diastolic blood pressure by about 5 mm Hg and thereby reducing systemic blood pressure in the subject.

12. The method of claim 11 wherein reducing said blood pressure is without a significant change in homocysteine blood levels.

13. (canceled)

14. A method for treating sexual dysfunction in a subject comprising administering to the subject a composition consisting essentially of an effective amount of folic acid or a folic acid active metabolite, thereby treating the sexual dysfunction.

15. The method of claim 14 wherein the sexual dysfunction is erectile dysfunction.

16-19. (canceled)

20. A composition comprising about 30 mg of folic acid in a pharmaceutically acceptable vehicle.

21. The composition of claim 20 further comprising a selected drug suitable for treating coronary heart disease.

22. The composition of claim 21 wherein the selected drug is selected from the group consisting of an ACE inhibitor, beta blocker, calcium channel blocker, nitrates and salicylates.

23. The composition of claim 20 comprising a tablet formulated in an orally acceptable dispersant.

24. The composition of claim 23, wherein the tablet is in combination with a cardiovascular drug selected from the group consisting of an ACE inhibitor, beta blocker, calcium channel blocker, nitrates and salicylates.

25. The composition of claim 23, wherein said tablet is a sustained tablet that provides a blood level of 5-methylene tetrahydrofolate effective to reduce diastolic blood pressure by at least 5 mm Hg in a subject having coronary heart disease.

26. A packaged formulation comprising a pharmaceutical composition comprising an amount of folic acid or a pharmaceutically acceptable salt, ester or prodrug thereof comprising about 20 to about 100 mg in a pharmaceutically acceptable vehicle and instructions for use in (1) reducing systemic blood pressure or (2) treating erectile dysfunction.

27. (canceled)

28. The method of claim 1 further comprising administering to a subject at risk of developing ischemic heart disease a daily dose of about 20 mg to about 100 mg folic acid to provide a sustained blood level of folic acid active metabolite that delays or minimizes development of ischemic heart disease, thereby prophylatically treating the subject at risk of developing ischemic heart disease.

29-30. (canceled)

31. The method of claim 28 wherein the subject at risk to develop ischemic heart disease has at least one risk factor selected from the group consisting of obesity, smoker, diabetics, high cholesterol, and high blood pressure.

32. (canceled)

33. The method of claim 1, wherein the intermediate metabolite of folic acid is folinic acid or a pharmaceutically acceptable salt thereof.

34. The method of claim 33 wherein the amount of folinic acid is 30 mg administered daily or 15 mg administered twice daily.
